SEQUENCE LISTING (5 sequences)

SEQ ID NO: 1 - DNA, 143 bp, synthetic construct
  source 1..143: mol_type = other DNA
ccaaatgtaactagatgctttcacaaaccccacccacaaagcagcacatgtttttaagacttcagttttctattcacatcggcctcataatacccaccctgacctgctgtaaaagacctggaacaaacaaaaatgattacacc

SEQ ID NO: 2 - DNA, 297 bp, synthetic construct
  source 1..297: mol_type = other DNA
tctaggccaccccttgctgattttacttcctcctttcctcacccacagcagtctggctgccagccacccttccaatgactctgttcttgtcaaggtcacgctgacctctgtatttacagatccagtggtagctccacacgccttaccttacttgaccttcctaatccatttggcactggggcccactgcttccttgaaactccctgttccattgaatctatccttctttccaactgtctcccacacaccactcctcagatgtccgatgagcacttcaaactctgtcaaaatgaactt

SEQ ID NO: 3 - DNA, 258 bp, synthetic construct
  source 1..258: mol_type = other DNA
acattaggtctcacattgcgtattctcaaggaggccaactaattctgtaaccatggataggtcctgtgataaattggaaaatgacttctggggcaacagtctctagttctcagaaaggaaattcacagaagtgagcggttggccagaaggccaaaaggccattgcctcagagtggatcccaaagcagagtgatgtggccattcacagcatgtttgagtatgtaagaaaaaagaaaaataagaaagggtatgatttctc

SEQ ID NO: 4 - DNA, 327 bp, synthetic construct
  source 1..327: mol_type = other DNA
atggggatttagtagctgagaaagagttgctgagtagtgagggtgggggcaggtcgtagagggccataattcctagaaaaagatgaccttgtccaattgagaggtagtgagaccagcaaacactggcaattaaaaattaacacaggccaaaagtgaaatcgatgcctaaatttgataggttttcaagaaaagtttatgacatgcagttcatagaaaagagaacattttctcagtgttcacttttaagatgccaaaggtcaatcttacttggaaagatccttccacagaaaagcagaaatgtacagggtctcctagcaaagtataagg

SEQ ID NO: 5 - DNA, 1025 bp, synthetic construct
  source 1..1025: mol_type = other DNA
tctaggccaccccttgctgattttacttcctcctttcctcacccacagcagtctggctgccagccacccttccaatgactctgttcttgtcaaggtcacgctgacctctgtatttacagatccagtggtagctccacacgccttaccttacttgaccttcctaatccatttggcactggggcccactgcttccttgaaactccctgttccattgaatctatccttctttccaactgtctcccacacaccactcctcagatgtccgatgagcacttcaaactctgtcaaaatgaacttacattaggtctcacattgcgtattctcaaggaggccaactaattctgtaaccatggataggtcctgtgataaattggaaaatgacttctggggcaacagtctctagttctcagaaaggaaattcacagaagtgagcggttggccagaaggccaaaaggccattgcctcagagtggatcccaaagcagagtgatgtggccattcacagcatgtttgagtatgtaagaaaaaagaaaaataagaaagggtatgatttctcatggggatttagtagctgagaaagagttgctgagtagtgagggtgggggcaggtcgtagagggccataattcctagaaaaagatgaccttgtccaattgagaggtagtgagaccagcaaacactggcaattaaaaattaacacaggccaaaagtgaaatcgatgcctaaatttgataggttttcaagaaaagtttatgacatgcagttcatagaaaagagaacattttctcagtgttcacttttaagatgccaaaggtcaatcttacttggaaagatccttccacagaaaagcagaaatgtacagggtctcctagcaaagtataaggccaaatgtaactagatgctttcacaaaccccacccacaaagcagcacatgtttttaagacttcagttttctattcacatcggcctcataatacccaccctgacctgctgtaaaagacctggaacaaacaaaaatgattacacc

(19) United States
(12) Patent Application Publication
(10) Pub. No.: US 2025/0198989 A1
(43) Pub. Date: Jun. 19, 2025

(54) METHOD FOR DETECTING THE BIOLOGICAL ACTIVITY OF RECOMBINANT HUMAN GROWTH HORMONE MEDICINE

(71) Applicant: National Institutes for Food and Drug Control, Beijing (CN)

(72) Inventors: Xiaoming Zhang, Beijing (CN); Jing Li, Beijing (CN); Chenggang Liang, Beijing (CN); Ping Lv, Beijing (CN)

(73) Assignee: National Institutes for Food and Drug Control, Beijing (CN)

(21) Appl. No.: 19/050,083

(22) Filed: Feb. 10, 2025

(30) Foreign Application Priority Data
Feb. 21, 2024 (CN) 2024101925520

(51) Int. Cl. G01N 33/50; C12N 5/071; C12N 15/86

(52) U.S. Cl. CPC G01N 33/5044; C12N 5/0686; C12N 15/86; C12N 2503/02; C12N 2510/00; C12N 2740/15043; G01N 2333/90241

(57) ABSTRACT

The embodiments of the present disclosure provide method for Detecting the Biological Activity of Recombinant Human Growth Hormone Medicine. The result of biological activity detection method for GH medicine provided in present disclosure is stable and reliable. The operation is simple. The experimental period is short. there is no need for cells or other components from human primary tissue sources. The specificity and accuracy are high. The method can be used for product development and release inspection. It avoids cell contamination and errors caused by long-term incubation and multi-step operations. The color result is stable and the quality is controllable. The biological activity of GH medicine detected by the method is correlated with clinical efficacy, providing important reference for the treatment of related diseases in clinical practice.

### CROSS-REFERENCE TO RELATED APPLICATIONS

This application is a continuation of claims priority of Chinese Patent Application No. 2024101925520, filed on Feb. 21, 2024, the contents of which are hereby incorporated by reference.

### TECHNICAL FIELD

The disclosure relates to the technical field of biomedicine, particularly a method for detecting the biological activity of recombinant human growth hormone medicine.

### BACKGROUND

Human growth hormon (GH) secreted by anterior pituitary gland is a peptide hormone consisting of 191 amino acids. It has various important physiological functions, such as stimulating metabolism, and promoting the development and maturation of tissues, organs, bones, and the entire human body etc. In addition, GH is closely related to aging and the occurrence of cancer. The chemical structure of recombinant human growth hormone medicine is identical to the growth hormone secreted by human pituitary gland, and the physiological and pharmacological effects thereof remain the same. It can significantly promote height growth in short children and improve the growth and development of various organs and tissues of their entire body.

The biological activity of recombinant human growth hormone medicine is closely related to the structure and function thereof. To evaluate its quality, biological activity detection is an effective and necessary means. At present, GH activity detection mainly relies on in vivo animal assay, which have disadvantages such as time consumption, complicated operation, high individual variability, high requirements for experimental operators, and the consumption of a large number of animals. In the United States Pharmacopeia 2023 edition, in vivo animal assay has been replaced by Nb2-11 cell proliferation assay for GH medicine release inspection. Nb2-11 cell proliferation assay has three shortcomings. Firstly, the detection time is long, which requires at least 30 hours from cell plating to obtaining experimental results. Secondly, the specificity is low, that is, the assay not only responds to GH, but also to human prolactin and interleukin-2. Thirdly, the assay is to detect the binding between GH and prolactin receptors, which cannot well reflect the mechanism of GH action. Therefore, there is an urgent need to develop new biological activity assay methods in the field.

### SUMMARY

One or more embodiments of the present disclosure provide a method for constructing a cell line for stably detecting the biological activity of GH medicine, wherein said method comprises:
a) transfecting plasmids stably expressing GHR, STAT5B, and 3H-IGF-1-P2-Luc reporter genes respectively into cells to obtain high titer lentivirus;
b) using the lentivirus stably expressing GHR and 3H-IGF-1-P2-Luc in step a) to infect target cell;
c) using the lentivirus stably expressing STAT5B in step a) to infect the target cell infected in step b) to obtain a cell line for stably detecting the biological activity of GH medicine.

The sequence of 3H is shown in SEQ ID NO: 5.

In some embodiments, the vector for stable expression of GHR in step a) is vector pLVX-IRES-Hyg.

In some embodiments, the plasmid for stable expression of GHR is pLVX-IRES-Hyg-Flag-GHR.

In some embodiments, the vector for stable expression of STAT5B in step a) is vector Plentic-C-mGFP.

In some embodiments, the plasmid for stable expression of STAT5B is pLenti-MYC-STAT5B-IRES-BLAST.

In some embodiments, the vector for stable expression of 3H-IGF-1-P2-Luc in step a) is vector pLV-Puro.

In some embodiments, the plasmid for stable expression of 3H-IGF-1-P2-Luc is pLV-Puro-3H-IGF-1-P2-LUC2P.

In some embodiments, the cells in step a) are HEK293 cells.

In some embodiments, the HEK293 cells are selected from HEK293LT cells.

In some embodiments, step a) further comprises the step of transfecting cells with plasmids for stably expressing GHR, STAT5B, and 3H-IGF-1-P2-Luc reporter genes which mix with plasmids pMD2.G and pSD, respectively.

In some embodiments, step a) further comprises the step of obtaining high titer lentivirus by ultracentrifugation after cell culture.

In some embodiments, the target cell in step b) is HepG2 cell.

In some embodiments, the target cell after infection in step b) is obtained through screening.

In some embodiments, the target cells after infection are screened by antibiotics.

In some embodiments, the antibiotics are selected from hygromycin and puromycin.

In some embodiments, the concentration of the hygromycin is 200 μg/mL.

In some embodiments, the concentration of puromycin is 2 μg/mL.

In some embodiments, the cell line for stably detecting the biological activity of GH medicine described in step c) is obtained through screening.

In some embodiments, antibiotics are used for screening.

In some embodiments, the antibiotics comprise blasticidin, hygromycin, and puromycin.

In some embodiments, step c) further comprises screening by limited dilution method in some embodiments after antibiotic treatment.

In some embodiments, the cell density of the limited dilution method is 1000 cells/mL.

In some embodiments, the cell concentration of the limited dilution method is 200 μL/well.

In some embodiments, step c) further comprises washing and culturing the cells after the limited dilution method.

In some embodiments, the cells are washed with PBS solution.

In some embodiments, the cells are cultivated using a culture medium containing human growth hormone but not fetal bovine serum.

In some embodiments, step c) further comprises rewashing the cultured cells and recultivating the cells.

In some embodiments, the culture medium for recultivation is EMEM without serum.

In some embodiments, the concentration of blasticidin is 5 μg/mL.

One or more embodiments of the present disclosure provide a method for stably detecting the biological activity of GH medicine, wherein said method comprises:
1) constructing a cell line stably expressing GHR, STAT5B, and 3H-IGF-1-P2-Luc reporter genes by the aforementioned method;
2) coincubating the cell line in step 1) with a GH medicine sample;
3) adding luciferase substrate and determining the biological activity of the GH medicine.

In some embodiments, the GH medicine sample in step 2) is a sample of diluted GH medicine.

In some embodiments, the dilution solution for the GH medicine sample is EMEM culture medium.

In some embodiments, the EMEM culture medium is an EMEM culture medium containing horse serum.

In some embodiments, the initial concentration of the GH medicine sample is 30-100 ng/mL.

In some embodiments, the initial concentration of the GH medicine sample is 60 ng/mL.

In some embodiments, the dilution ratio of the GH medicine sample is 1:1.

In some embodiments, the temperature for coincubation is 37° C. in step 2).

In some embodiments, the time for coincubation is 2.5-3 h in step 2).

In some embodiments, the time for coincubation is 2.5 h in step 2).

In some embodiments, the cell seeding density is 1-4×10⁴ cells/well in step 2).

In some embodiments, the cell seeding density is 4×10⁴ cells/well in step 2).

In some embodiments, a four-parameter curve is fitted according to the measured chemiluminescence values to determine the biological activity of the GH medicine sample in step 3).

In some embodiments, the four-parameter curve is obtained by reading the relative light unit values of chemiluminescence using microplate reader and fitting the data.

In some embodiments, the relative potency of the GH medicine sample is obtained by comparing the EC50 values of the four-parameter curves of the GH medicine samples.

One or more embodiments of the present disclosure provide a cell line for stably detecting the biological activity of GH medicine, wherein said cell line is constructed by the aforementioned method.

One or more embodiments of the present disclosure provide a product for stably detecting the biological activity of GH medicine, wherein said product comprises the aforementioned cell line.

In some embodiments, the product further comprises diluent and luciferase substrate.

In some embodiments, the diluent comprises EMEM culture medium and horse serum.

In some embodiments, the product further comprises instruction manual, positive control substance, and negative control substance.

In some embodiments, the product further comprises one or more sterile containers.

In some embodiments, the product comprises a reagent kit.

One or more embodiments of the present disclosure provide the aforementioned cell line for use in detecting the biological activity of GH medicine.

One or more embodiments of the present disclosure provide the aforementioned method for use in quality control of GH medicine production. The advantages and beneficial effects of the present disclosure:

The result of biological activity detection method for GH medicine provided in present disclosure is stable and reliable. The operation is simple. The experimental period is short. there is no need for cells or other components from human primary tissue sources. The specificity and accuracy are high. The method can be used for product development and release inspection. It avoids cell contamination and errors caused by long-term incubation and multi-step operations. The color result is stable and the quality is controllable. The biological activity of GH medicine detected by the method is correlated with clinical efficacy, providing important reference for the treatment of related diseases in clinical practice.

### BRIEF DESCRIPTION OF THE DRAWINGS

FIG. 1 shows the screening chart of IGF-1 gene enhancer sequence;

FIG. 2 shows optimization graph for different initial dilution concentrations;

FIG. 3 shows optimization graph for different incubation times;

FIG. 4 shows optimization graph for different cell densities;

FIG. 5 shows result graph of specificity verification

### DETAILED DESCRIPTION OF THE EMBODIMENTS

The following provides definitions of some terms used in the present disclosure. Unless otherwise specified, all technical and scientific terms used in the present disclosure generally have the same meaning as those commonly understood by ordinary persons skilled in the art to which the present disclosure belongs.

The present disclosure provides a method for constructing a cell line for stably detecting the biological activity of GH medicine, wherein said method comprises:
a) transfecting plasmids stably expressing GHR, STAT5B, and 3H-IGF-1-P2-Luc reporter genes respectively into cells to obtain high titer lentivirus;
b) using the lentivirus stably expressing GHR and 3H-IGF-1-P2-Luc in step a) to infect target cell;
c) using the lentivirus stably expressing STAT5B in step a) to infect the target cell infected in step b) to obtain a cell line for stably detecting the biological activity of GH medicine.

In some embodiments, biological activity refers to the specific ability or potential of a biological product to achieve a detected biological effect, and the corresponding biological activity of the biological product can be evaluated using the biological effect of a specific cell line.

In some embodiments, the vectors expressing GHR, STAT5B, and 3H-IGF-1-P2-Luc reporter genes include but are not limited to pLV-Puro, pLVX-IRES-Hyg, Plenti-C-mGFP, pCDH-EF1-Luc2-T2A-tdTomato, pCDH-MSCV-MCS-EF1-Puro, pCDH-MSCV-MCS-EF1-copGFP, pLVX-ZsGreen1-C1, pAdEasy-1, pShuttle-CMV, pShuttle, pAdTrack, pAdTrack-CMV, pShuttle-IRES-hrGFP-1, pShuttle-IRES-hrGFP-2, pShuttle-CMV-lacZ, pShuttle-CMV-EGFP-C, pXC1, pBHGE3, pAAV-MCS, pAAV-RC, pHelper, pAAV-LacZ, pLK0.1-puro, pLK0.1-CMV-tGFP, pLKO.1-puro-CMV-tGFP, pLK0.1-CMV-Neo, pLK0.1-Neo, pLKO.1-Neo-CMV-tGFP, pLKO.1-puro-CMV-TagCFP, pLKO.1-puro-CMV-TagYFP, pLKO.1-puro-CMV-TagRFP, pLKO.1-puro-CMV-TagFP635, pLKO.-puro-UbC-TurboGFP, pLKO.1-puro-UbC-TagFP635, pLKO-puro-IPTG-1×LacO, pLKO-puro-IPTG-3×LacO, pLP1, pLP2, pLP/VSV-G, pENTR/U6, pLenti6/BLOCK-iT-DEST, pLenti6-GW/U6-laminshrna, pcDNAI, 2/V5-GW/lacZ, pLenti6.2/N-Lumio/V5-DEST, pGCSIL-GFP and Lenti6.2/N-Lumio/V5-GW/lacZ.

In specific embodiments, the vector for stable expression of GHR is vector pLVX-IRES-Hyg, the vector for stable expression of STAT5B is vector Plentic-C-mGFP, and the vector for stable expression of 3H-IGF-1-P2-Luc is vector pLV-Puro.

In some embodiments, the plasmids are known as recombinant plasmids, which refer to the plasmids are artificially constructed on the basis of natural plasmids to adapt to laboratory operations. For example, in the present disclosure, the plasmid stably expressing GHR is plasmid pLVX-IRES-Hyg-Flag-GHR; the plasmid stably expressing STAT5B is plasmid pLenti-MYC-STAT5B-IRES-BLAST; the plasmid stably expressing 3H-IGF-1-P2-Luc is plasmid pLV-Puro-3H-IGF-1-P2-LUC2P.

In some embodiments, the cells are HEK293 cells, including but not limited to HEK293LT cells, HEK293T cells, 293T/17 cells, 293T/17SF cells, 293H cells, 293A (adherent) cells, 293E cells, 293-6E cells, 293F cells, 293 FT cells, 293FTM cells, 293S (suspension) cells, 293SG cells, and 293SGGD cells.

In specific embodiments, the HEK293 cells are selected from HEK293LT cells.

In some embodiments, the target cell includes but are not limited to HepG2 cell, Raji cell, WIL2-S cell, SU-DHL-10 cell, DOHH2 cell, BJAB cell, A20 cell, SU-DHL-4 cell, P30/OHK cell, P116 cell, MUTZ-3 cell, SUPB15 cell, SNK6 cell, SK-MEL-28 cell, C8161 cell, and RPMI-7951 cell.

In specific embodiments, the target cell is HepG2 cell.

In some embodiments, the cell line is a HepG2 target cell that stably expresses GHR, STAT5B, and 3H-IGF-1-P2-Luc reporter genes.

The target cell after infection in step b) is obtained through screening.

The target cells after infection are screened by antibiotics.

In some embodiments, the antibiotics include but are not limited to Blasticidin, Hygromycin B, Puromycin, 6-thioguanine, and Ampicillin.

In specific embodiments, the antibiotics are selected from hygromycin and puromycin.

The present disclosure provides a method for stably detecting the biological activity of GH medicine, wherein said method comprises:
1) constructing a cell line stably expressing GHR, STAT5B, and 3H-IGF-1-P2-Luc reporter genes using the aforementioned method;
2) incubating the cell line in step 1) with a GH medicine sample;
3) adding luciferase substrate and determining the biological activity of the GH medicine.

A four-parameter curve is fitted according to the measured chemiluminescence values to determine the biological activity of the GH medicine sample in step 3).

The four-parameter curve is obtained by reading the relative light unit values of chemiluminescence using microplate reader and fitting the data.

The relative potency of the GH medicine sample is obtained by comparing the EC50 values of the four-parameter curves of the GH medicine samples.

In some embodiments, luciferase derives from bioluminescent organisms in nature. [98] It is a general term for enzymes that can catalyze the oxidation of luciferin or fatty aldehydes to emit light in living organisms. According to the source of different kinds of organisms, luciferase can be divided into firefly luciferase (FL) and bacterial luciferase (BL). At present, the luciferase gene derived from North American fireflies is the most widely used, which can encode a luciferase protein consisting of 550 amino acids. FL gene derives from fireflies and catalyzes the oxidation and decarboxylation of D-luciferin with the participation of Mg²⁺, ATP, and O₂, producing activated oxidized luciferin and emitting photons, and producing fluorescence at 550-580 nm.

Luciferase reporter gene detection is an important tool to analyze interaction relationship of the potential cis elements (such as promoters, enhancers, and silencers) and trans acting factors in the lateral regions of structural genes in modern molecular biology research. The luciferase reporter gene system is a reporting system that uses luciferin as a substrate to detect the activity of firefly luciferase. Luciferase can catalyze the oxidation of Luciferin to Oxyluciferin. During the oxidation of Luciferin, bioluminescence is emitted, which is then measured using a chemiluminescence or liquid scintillation analyzer.

In some embodiments, the relative light unit values of chemiluminescence is read using microplate reader.

In some embodiments, the value of chemiluminescence is determined by a luciferase assay kit.

In some embodiments, the value of chemiluminescence is determined by Promega's Steady Glo luciferase assay system. The substrate of luciferase is oxidized under the catalysis of luciferase, and emits biological fluorescence which can be detected by chemiluminescence apparatus during the oxidation process.

In some embodiments, a four-parameter curve refers to a curve fitted according to the four-parameter equation Y=Bottom+(Top−Bottom)/(1+10\hat{ }((Log EC50−X)*HillSlope)), which can provide four parameters including Top, Bottom, EC50, and HillSlope.

The present disclosure provides a product for stably detecting the biological activity of GH medicine, wherein said product comprises the aforementioned cell line.

The product further comprises diluent and luciferase substrate.

The product further comprises instruction manual, positive control substance, and negative control substance.

The product further comprises one or more sterile containers. Such containers can be boxes, ampoules, bottles, vials, tubes, bags, pouches, blister packs, or other suitable container forms known in the art. Such containers can be made of plastic, glass, laminated paper, metal foil, or other materials.

Below is a detailed explanation of the present disclosure with specific implementation examples. The following examples will be helpful for technical person skilled in the art to understand the present disclosure, but do not limit the present disclosure in any form.

### Example 1 Screening Regulatory Sequences of Report Genes

### 1. Experimental Materials

HepG2 cells (ATCC, item number HB-8065), EMEM culture medium (ATCC, item number 30-2003, batch number 2042333), fetal bovine serum (Gibco, item number 10082, batch number 2236255CP), phosphate buffer solution (Gibco, item number 20012, batch number 2277093), pGL4.21 [luc2P/Puro] (Promega, item number E6761), pCDH-EF1-MCS-CMV-copGFP-T2A-Puro (Synbio Technologies, Suzhou, China), pcDNA3.1 (Wuhan GeneCreate Biological Engineering Co., Ltd. Wuhan, China), Lipofectamine 3000 transfection reagent (Invitrogen, item number L3000-001, batch number CN2481201), Recombinant human growth hormone national standard (National Institute for Food and Drug Control, China, batch number 140635-202206), Steady-Glo® Luciferase reporter gene detection system (Promega, batch number 0000545352), OPTI-MEM (Gibco, item number 31985-062, batch number 2042333).

### 2. Experimental Methods

1) plasmid construction: IGF-1 gene regulatory sequence, STAT5B-myc, and GHR gene were ligated to plasmid vector pGL4.21 [luc2P/Puro], pCDH-EF1-MCS-CMV-copGFP-T2A-Puro, and pcDNA3.1s, respectively.
2) HepG2 cells were inoculated into a 12-well plate. When a confluence of 70-90% was reached, the culture medium was replaced with Opti MEM culture medium, and GHR, STAT5B, and reporter gene vector plasmids of which a concentration of is 1.0 μg for each plasmid were transfected with lipofectamine 3000 transfection reagent plasmid.
3) The cells were seeded into a 24-well plate after transfection with plasmids for 24 hours. After the cells adhered overnight, they were washed with PBS solution and the culture medium was replaced with experimental culture medium containing 200 ng/mL human growth hormone without fetal bovine serum. The control well was replaced with experimental culture medium without human growth hormone.
4) after incubation for 24 hours, 500 μl of luciferase substrate was added to each well, and the cells were lysed at room temperature for 15 minutes. After standing for 15 minutes, the fluorescence signal value was read.

Four reporter genes used in the experiment were H17, H75-76, and H77-78 which are derived from the enhancer region of IGF-1 gene, and 3H which is the combination of these three regulatory regions, respectively. The promoters of the four plasmids are all promoter P2 of IGF-1 (IGF1-P2). The sequences are as follows:

IGF1-P2 sequence of promoter P2 of IGF-1 (143 bp)
(SEQ ID NO: 1)
ccaaatgtaactagatgctttcacaaaccccacccacaaagcagcacatgtttttaagacttcagttttctattcac
atcggcctcataatacccaccctgacctgctgtaaaagacctggaacaaacaaaaatgattacacc
H17 sequence (297 bp)
(SEQ ID NO: 2)
tctaggccaccccttgctgattttacttcctcctttcctcacccacagcagtctggctgccagccacccttccaat
gactctgttcttgtcaaggtcacgctgacctctgtatttacagatccagtggtagctccacacgccttaccttacttgaccttcct
aatccatttggcactggggcccactgcttccttgaaactccctgttccattgaatctatccttctttccaactgtctcccacacac
cactcctcagatgtccgatgagcacttcaaactctgtcaaaatgaactt
H77-78 sequence (258 bp)
(SEQ ID NO: 3)
acattaggtctcacattgcgtattctcaaggaggccaactaattctgtaaccatggataggtcctgtgataaattg
gaaaatgacttctggggcaacagtctctagttctcagaaaggaaattcacagaagtgagcggttggccagaaggccaaaa
ggccattgcctcagagtggatcccaaagcagagtgatgtggccattcacagcatgtttgagtatgtaagaaaaaagaaaaat
aagaaagggtatgatttctc
H75-76 sequence (327 bp)
(SEQ ID NO: 4)
atggggatttagtagctgagaaagagttgctgagtagtgaggggggggcaggtcgtagagggccataattc
ctagaaaaagatgaccttgtccaattgagaggtagtgagaccagcaaacactggcaattaaaaattaacacaggccaaaag
tgaaatcgatgcctaaatttgataggttttcaagaaaagtttatgacatgcagttcatagaaaagagaacattttctcagtgttca
cttttaagatgccaaaggtcaatcttacttggaaagatccttccacagaaaagcagaaatgtacagggtctcctagcaaagta
taagg
H17+H77-78+H75-76+IGF-1 P2 sequence (3H, 1025 bp)
(SEQ ID NO: 5)
tctaggccaccccttgctgattttacttcctcctttcctcacccacagcagtctggctgccagccacccttccaat
gactctgttcttgtcaaggtcacgctgacctctgtatttacagatccagtggtagctccacacgccttaccttacttgaccttcct
aatccatttggcactggggcccactgcttccttgaaactccctgttccattgaatctatccttctttccaactgtctcccacacac
cactcctcagatgtccgatgagcacttcaaactctgtcaaaatgaacttacattaggtctcacattgcgtattctcaaggaggc
caactaattctgtaaccatggataggtcctgtgataaattggaaaatgacttctggggcaacagtctctagttctcagaaagga
aattcacagaagtgagcggttggccagaaggccaaaaggccattgcctcagagtggatcccaaagcagagtgatgtggc
cattcacagcatgtttgagtatgtaagaaaaaagaaaaataagaaagggtatgatttctcatggggatttagtagctgagaaa
gagttgctgagtagtgagggtgggggcaggtcgtagagggccataattcctagaaaaagatgaccttgtccaattgagag
gtagtgagaccagcaaacactggcaattaaaaattaacacaggccaaaagtgaaatcgatgcctaaatttgataggttttcaa
gaaaagtttatgacatgcagttcatagaaaagagaacattttctcagtgttcacttttaagatgccaaaggtcaatcttacttgga
aagatccttccacagaaaagcagaaatgtacagggtctcctagcaaagtataaggccaaatgtaactagatgctttcacaaa
ccccacccacaaagcagcacatgtttttaagacttcagttttctattcacatcggcctcataatacccaccctgacctgctgtaa
aagacctggaacaaacaaaaatgattacacc

### 3. Experimental Results

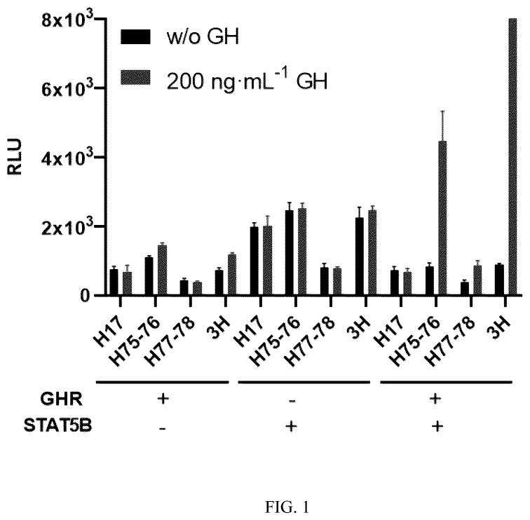

HepG2 cells transfected with the reporter gene and GHR alone or with the reporter gene and STAT5B alone did not activate the expression of the reporter gene after 24 hours of GH stimulation; HepG2 cells co-transfected with GHR, STAT5B, and reporter genes showed a 12-fold increase in luciferase expression induced by the plasmid containing 3H after 24 hours of GH stimulation compared to the control without GH. However, H17 and H77-78 cannot induce the expression of luciferase when co-expressing GHR and STAT5B. H75-76 can initiate luciferase expression (a 5-fold increase), but its ability is lower than that of 3H (FIG. 1).

Conclusion: IGF-1 gene enhancers H17 and H77-78 cannot induce luciferase expression, while H75-76 can induce luciferase expression under the premise of the co-expression of GHR and STAT5B, but the effect is significantly lower than 3H, the combination of the three regions. Therefore, 3H was selected for the construction of lentiviral plasmid.

### Example 2 A Method for Stable and Rapid Detecting of the Biological Activity of GH Medicine

### 1. Experimental Materials

EMEM culture medium (ATCC, item number 30-2003, batch number 2042333), fetal bovine serum (Gibco, item number 10082, batch number 2236255CP), horse serum (Gibco, item number 26050, batch number 1763585), phosphate buffer solution (Gibco, item number 20012, batch number 2277093), recombinant human growth hormone national standard (National Institute for Food and Drug Control, China, batch number 140635-202206), bovine placental prolactin (BPL, GLPBIO, item number GP20835, batch number GP208351); Interleukin-2 (IL-2, Gibco, item number PHC0027, batch number 2258434) Steady-Glo.®. Luciferase reporter gene detection system (Promega, item number E2548, batch number 0000545352), 96-well white cell culture plate (PerkinElmer, batch number 8013-21281), HEK293LT cells, pLV-Puro, pLVX-IRES-Hyg, and Plentic-C-mGFP plasmids purchased from Sino Biological Inc (Beijing, China).

### 2. Experimental Methods

(1) Construct HepG2 target cells stably expressing GHR, STAT5B, and 3H-IGF-1-P2-Luc reporter genes.
(a) The gene regulatory sequences selected in pre-experiments were combined, which simulate better the mechanism and efficacy of human growth hormone. That is, IGF-1 gene promoter and expression regulatory sequence 3H-IGF-1-P2, along with luciferase gene Luc2P were ligated to vector pLV-Puro to obtain pLV-Puro-3H-IGF-1-P2-LUC2P.
(b) Flag-GHR was ligated to vector pLVX-IRES-Hyg to obtain pLVX-IRES-Hyg-Flag-GHR. STAT5B-myc was ligated to vector Plenti-C-mGFP to obtain pLenti-MYC-STAT5B-IRES-BLAST.
(c) Plasmid pLVX-IRES-Hyg-Flag-GHR, plasmid pLV-Puro-3H-IGF-1-P2-LUC2P, and plasmid pLenti-MYC-STAT5B-IRES-BLAST were mixed with plasmid pMD2. G and pSD, respectively, and were transfected into HEK293LT cells. After the cells were cultured 48-72 hours, the cell supernatant was collected by centrifugation. The cell supernatant was filtered by a 0.45 μm sterile filter, and high titer lentivirus was obtained after ultracentrifugation.
(d) HepG2 cells (target cells) were infected with pLVX-IRES-Hyg-Flag-GHR and pLV-Puro-3H-IGF-1-P2-LUC2P lentiviruses.
(e) After 2 days of cultivation, a mixed clone cell library was obtained by screening with hygromycin and puromycin at concentrations of 200 μg/mL and 2 μg/mL, respectively.
(f) The mixed clone cell library obtained in step (e) was infected with pLenti-MYC-STAT5B-IRES-BLAST lentivirus.
(g) After 2 days of cultivation, a mixed clone cell library was obtained by screening with the addition of blasticidin hygromycin, and puromycin. The concentration of blasticidin was 5 μg/mL, the concentrations of hygromycin and puromycin were 200 μg/mL and 2 μg/mL, respectively.
(h) monoclonal cell line was screened by limited dilution method. The dilution method is as follows: the mixed cloned cells obtained in step (g) were diluted to 1000 cells/mL, were added well A1 of a transparent 96-well plate with 200 μL/well, and then the concentration was diluted at a ratio of 2 times from well A1 to well H1. For example, 100 μL of cell suspension was taken from well A1 and added to well B1, then 100 μL of culture medium was added therein. After mixing, 100 μL of cell suspension was taken and added to well C1, then 100 μL of culture medium was added therein. After mixing the first column, the concentration was diluted at a ratio of 2 times from the first column to the twelfth column. The plate was placed in incubator for cultivation, avoiding movement during the time. After 3 days, the growth status of the cells was observed continuously and the position of the monoclonal cell was marked on the well plate, and monoclonal cell line was cultured to be expanded.
(i) Positive clone cell line was obtained through pre-experiment and flow cytometry screening. The pre-experiment method is as follows: The monoclonal cells obtained in (h) were seeded into a 24-well plate and adherent culture overnight, and then washed once with PBS solution. The culture medium was replaced with experimental culture medium containing 200 ng/mL human growth hormone without fetal bovine serum, while the culture medium in the control well was replaced with experimental culture medium without human growth hormone. After incubation for 6 hours, 500 μl of luciferase substrate was added to each well, and the cells were lysed at room temperature for 15 minutes. After standing for 15 minutes, the fluorescence signal value was read. The monoclonal cell line that induces luciferase expression in response to GH stimulation was selected. Then the cells were diluted to 500000 cells/mL, with washing twice with PBS solution. The culture medium was replaced with serum-free EMEM. The cells were cultured overnight, and collected for flow cytometry. The monoclonal cell line with high GHR positivity rates was selected for subsequent experiments.
(2) The GH samples were pre-diluted initial concentrations to 100 ng/ml, 60 ng/mL, and 30 ng/mL, and then diluted in equal proportions using EMEM culture medium containing horse serum. The dilution ratio was 1:1, resulting in a total of 10 concentrations of GH.
(3) After the target cells were washed twice with PBS solution, then resuspended in EMEM culture medium containing horse serum, and filtered by a 100 μl cell filter, and diluted to 8×10⁵ cells/mL using EMEM culture medium containing horse serum. The cells were inoculated into a 96-well white cell culture plate with 50 μl/well and cultured overnight (18 h-20 h). The number of the target cells is 40000 per well. The GH sample at a concentration of 50 μl/well diluted in step (2) was added into the target cells with 3 replicates per concentration. The cells were incubated at 37° C. for 2.5 hours in incubator.
(4) Luciferase substrate was added, and then a four-parameter curve was fitted based on the measured chemiluminescence values to detect the biological activity of the antibody.
(5) Specificity verification of the method: GH, IL-2, and BPL were pre-diluted initial concentration to 3 nM, and then diluted in equal proportions using EMEM culture medium containing horse serum. The dilution ratio was 1:1, resulting in a total of 10 concentrations of GH. The biological activity of GH, IL-2, and BPL were detected according to steps (3) to (4).

### 3. Experimental Results

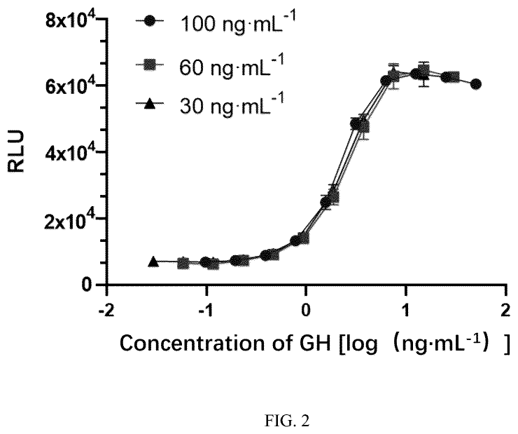

The experiment was conducted with three initial concentrations of GH, 100 ng/mL, 60 ng/mL, and 30 ng/ml, diluted 1:1 for 10 gradients, and a cell density of 40000 cells per well to optimize the initial concentration of GH. The results showed that the distribution of S-shaped curve points obtained from an initial concentration of 60 ng/mL was the most uniform, and the correlation coefficient R² was the highest. Therefore, the optimal initial concentration for the method was determined to be 60 ng/ml (FIG. 2).

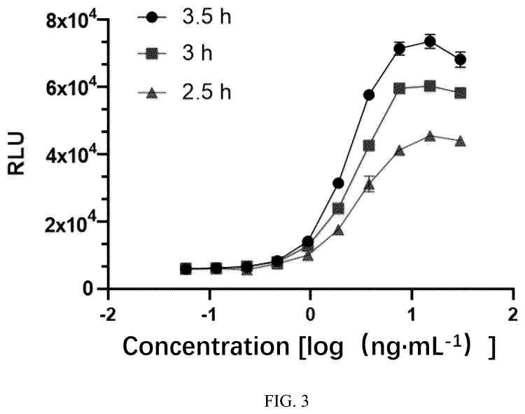

The experiment was conducted with an initial concentration of 60 ng/mL GH, diluted 1:1 for 10 gradients, and a cell density of 40000 cells per well. The effects of exposure times of 2.5, 3, and 3.5 hours were detected. S-type proliferation curves could be obtained by the three exposure times, respectively. The RLU value increased with the prolongation of exposure time. However, the correlation coefficient R² of the curve decreased with the prolongation of exposure time. Taking into account the RLU value and R², the optimal operating time for the method was determined to be 2.5 hours (FIG. 3).

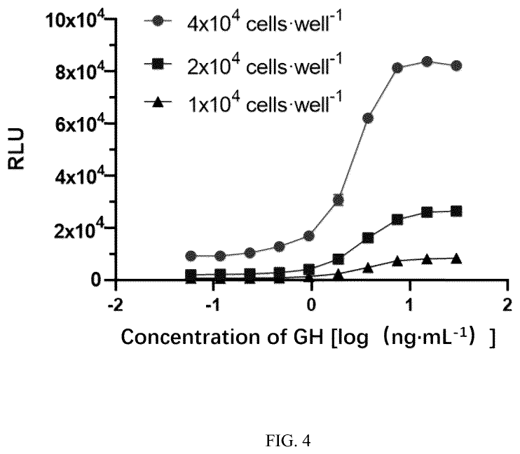

Next, the cell density was optimized by using 60 ng/mL GH as the initial dilution concentration diluted 1:1 for 10 gradients. The cell density was adjusted to 8×10⁵, 4×10⁵, and 2×10⁵ cells per milliliter, respectively. The cells were added into a 96-well plate (with cell densities of 4×10⁴, 2×10⁴, and 1×10⁴ cells per well) with 50 μl per well. As shown in FIG. 4, S-type proliferation curves could be obtained by the three cell densities. the signal-to-noise ratio of curve fitted by 4-parameter of 40000 (4×10⁴) cells per well was the highest. The optimal cell density for the method was determined to be 40000 cells/well.

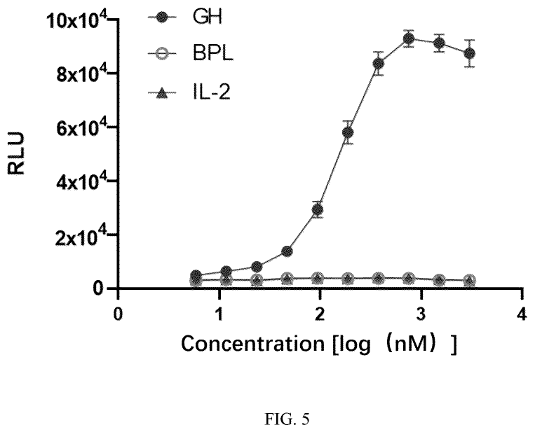

The result of specificity validation of the method showed that the method had high specificity for detecting GH biological activity. IL-2 and BPL at the same molar concentration could not stimulate cells to produce luciferase (FIG. 5).

Conclusion: The method for stable and rapid detecting of the biological activity of GH medicine had high specificity and no response to IL-2 and BPL. The method conditions after optimizing are: initial dilution concentration of GH is 60 ng/ml, dilution ratio is 1:1, dilution gradients are 10, cell density seeded is 40000 cells/well, and GH incubation time is 2.5 hours.

The above embodiments are only used to understand the methods and core ideas of the present disclosure, and do not constitute limitations on the present disclosure. It should be pointed out that for ordinary technical person skilled in the art, without departing from the principles of the present specification, the present specification can be modified by several improvements and modifications, and these improvements and modifications will further fall within the scope of protection of the present claims and the present specification.

This application contains a Sequence Listing XML as a separate part of the disclosure, set forth in accordance with the requirements of 37 CFR-1.831-1.835. The XML file named “CNUS-RB-U-2-2025-NIFDC sequences.xml”, created Feb. 10, 2025, 7,392 bytes in size, is submitted herewith and is incorporated by reference in its entirety.

What is claimed is:

1. A method for constructing a cell line for stably detecting the biological activity of GH medicine, wherein said method comprises:
a) transfecting plasmids stably expressing GHR, STAT5B, and 3H-IGF-1-P2-Luc reporter genes respectively into cells to obtain high titer lentivirus;
b) using the lentivirus stably expressing GHR and 3H-IGF-1-P2-Luc in step a) to infect target cell;
c) using the lentivirus stably expressing STAT5B in step a) to infect the target cell infected in step b) to obtain a cell line for stably detecting the biological activity of GH medicine;
the sequence of 3H-IGF-1-P2 is shown in SEQ ID NO: 5;
the target cell in step b) is HepG2 cell;
the vector for stable expression of GHR in step a) is vector pLVX-IRES-Hyg;
the vector for stable expression of STAT5B in step a) is vector Plentic-C-mGFP;
the vector for stable expression of 3H-IGF-1-P2-Luc in step a) is vector pLV-Puro.

2. The method according to claim 1, wherein the plasmid for stable expression of GHR is pLVX-IRES-Hyg-Flag-GHR.

3. The method according to claim 1, wherein the plasmid for stable expression of STAT5B is pLenti-MYC-STAT5B-IRES-BLAST.

4. The method according to claim 1, wherein the plasmid for stable expression of 3H-IGF-1-P2-Luc is pLV-Puro-3H-IGF-1-P2-LUC2P.

5. The method according to claim 1, wherein the cells in step a) are HEK293 cells.

6. The method according to claim 1, wherein step a) further comprises the step of transfecting cells with plasmids for stably expressing GHR, STAT5B, and 3H-IGF-1-P2-Luc reporter genes which mix with plasmids pMD2.G and pSD, respectively.

7. The method according to claim 6, wherein step a) further comprises the step of obtaining high titer lentivirus by ultracentrifugation after cell culture.

8. The method according to claim 1, wherein the target cell after infection in step b) is obtained through screening.

9. The method according to claim 8, wherein the target cells after infection are screened by antibiotics.

10. The method according to claim 1, wherein the cell line for stably detecting the biological activity of GH medicine described in step c) is obtained through screening.

11. The method according to claim 10, wherein antibiotics are used for screening.

12. The method according to claim 11, wherein step c) further comprises screening by limited dilution method after antibiotic treatment.

13. The method according to claim 12, wherein step c) further comprises washing and culturing the cells after the limited dilution method.

14. A method for stably detecting the biological activity of GH medicine, wherein said method comprises:
1) constructing a cell line stably expressing GHR, STAT5B, and 3H-IGF-1-P2-Luc reporter genes by the method according to claim 1;
2) coincubating the cell line in step 1) with a GH medicine sample;
3) adding luciferase substrate and determining the biological activity of the GH medicine.

15. The method according to claim 14, wherein the GH medicine sample in step 2) is a sample of diluted GH medicine.

16. A cell line for stably detecting the biological activity of GH medicine, wherein said cell line is constructed by the method according to claim 1.

17. A product for stably detecting the biological activity of GH medicine, wherein said product comprises the cell line according to claim 16.

18. The product according to claim 17, wherein the product further comprises diluent and luciferase substrate.

19. The product according to claim 17, wherein the product further comprises instruction manual, positive control substance, and negative control substance.

20. The product according to claim 17, wherein the product further comprises one or more sterile containers.
